(19) United States
(12) Patent Application Publication
(10) Pub. No.: US 2023/0380978 A1
(43) Pub. Date: Nov. 30, 2023

(54) Implantable Partial Temporomandibular Joint Prosthesis, Corresponding Production Method, and Temporomandibular Joint Prosthesis

(71) Applicant: KARL LIEBINGER MEDIZINTECHNIK GMBH & CO. KG, Mühlheim/Donau (DE)

(72) Inventors: Adem Aksu, VS-Schwenningen (DE); Ralf Leibinger, Mühlheim a.d. Donau (DE); Frank Reinauer, Emmingem-Liptingen (DE); Tobias Wolfram, Dreieich (DE)

(21) Appl. No.: 18/248,908

(22) PCT Filed: Oct. 26, 2021
(86) PCT No.: PCT/EP2021/079734

§ 371 Date: Apr. 13, 2023

(30) Foreign Application Priority Data
Oct. 27, 2020 (DE) 10 2020 213 508.8

(51) Int. Cl. A61F 2/30

(52) U.S. Cl. CPC A61F 2/3099; A61F 2/3094; A61F 2002/30014

(57) ABSTRACT

The invention relates to an implantable partial temporomandibular joint prosthesis (90, 90′, 90″) having a first implant part (10, 10′, 10″) that can be fastened to a lower jaw (40) and has a condyle (42, 42′, 42″) with respect to which a joint surface (52, 82) situated on a cranium (50) forms an abutment; wherein the first implant part (10, 10′, 10″) comprises a first component (20, 20′, 20″) formed from a metal material, which first component can be fastened to the lower jaw (40), and a second component (30, 30′, 30″) formed from a plastics material, which second component comprises the condyle (42, 42′, 42″); and wherein the first component (20, 20′, 20″) is provided with a first connection piece (22, 22′, 22″) and the second component (30, 30′, 30″) is provided with a second connection piece (32, 32′, 32″), and the first connection piece (22, 22′, 22″) and the second connection piece (32, 32′, 32″) can be or are

### BACKGROUND

The present invention relates to an implantable partial temporomandibular joint prosthesis having a first implant part which can be fastened to a lower jaw and has a condyle with respect to which a joint surface arranged on a cranium forms an abutment; wherein the first implant part has a first component made of a metal material, which first component can be fastened to the lower jaw, and a second component made of a ceramic or a plastic, which second component has the condyle. The invention further relates to a corresponding method for producing an implantable temporomandibular joint prosthesis, and to an implantable temporomandibular joint prosthesis which comprises the partial temporomandibular joint prosthesis.

The temporomandibular joint (TMJ) is one of the most used and most important joints in the human body. It plays an essential role in guiding the movement of the mandible (lower jaw), in chewing, speaking, swallowing, and stress management. It is constantly in motion even during sleep, on account of the swallowing movement. To enable the temporomandibular joint to perform its movement sequences, the shape of the joint surfaces, the condition of the teeth, the position of the teeth, the shape of the teeth and the masticatory muscles have to form a functional system, which is subject to a certain degree of susceptibility to malfunction on account of the large number of components. The left and the right temporo-mandibular joints always work together and in this sense form a functional unit.

However, if disorders arise in the region of the temporomandibular joint, e.g. functional restrictions or diseases, this can lead to considerable impairment in the quality of everyday life of patients. Restoration of correct joint structures is usually only possible by surgery, i.e. by resection of the damaged joint and its replacement with a temporomandibular joint prosthesis.

A temporomandibular joint prosthesis is known, for example, from EP 3 003 225 B1, which provides a mechanism that permits a combined translational and rotational movement between skull and mandible, in which a sliding movement between surfaces takes place. However, wear and tear can occur.

### SUMMARY OF THE DISCLOSURE

One object of the present invention is to create an improved implantable partial temporomandibular joint prosthesis, a corresponding production method and a corresponding temporomandibular joint prosthesis, by means of which the sliding movement of the surfaces is carried out as reliably and safely as possible and at the same time with little wear, and in which the condyle can be suitably adapted to the individual circumstances of the TMJ with minimal effort.

The object is achieved by an implantable partial temporomandibular joint prosthesis having the features of claim 1. The solution according to the invention is therefore in particular that, in the case of a partial temporomandibular joint prosthesis of the type mentioned at the outset, the first component is provided with a first connection piece and the second component is provided with a second connection piece, and the first connection piece and the second connection piece are connectable or connected to each other by form-fit engagement at least in part.

With the implantable partial temporomandibular joint prosthesis according to the invention, a reliable slide pairing is thus provided for the individual joint surface forming the abutment, by virtue of the fact that the first implant part is provided with several components which engage in each other with their connection pieces and establish a form-fit connection using these, as a result of which they are reliably connectable or connected to each other. On the TMJ, the partial prosthesis forms, together with the articular surface (fossa), a slide pairing that allows a 5-axis movement of the temporomandibular joint (TMJ), wherein the condyle, as the rolling element, can be individually configured.

In order to achieve a particularly durable connection between the components of the partial temporomandibular joint prosthesis according to the invention, in a preferred development one of the connection pieces can be designed with at least one projection which engages in at least one complementary recess on the other connection piece.

In this case, the projection and the recess can produce the form fit in question on mutually opposite surfaces. In this case, a plurality of projections and recesses can also be formed on the components, it being possible for each of the components to be provided both with projections and with recesses.

For a particularly durable coupling of the connection pieces, and therefore of the components to each other, in a further development the at least one projection of one connection piece and the at least one complementary recess of the other connection piece can produce an all-round form fit. In this case, all-round form fits can be produced by individual, multiple or all connection pieces forming a connection to an assigned connection piece of the respective other components.

In a development that further strengthens the fastening of the connection pieces of the respective components, the first component and the second component can be fastened to each other by at least one connection element arranged as third component between the respective connection pieces. This third component therefore serves to lock the condyle position. On the one hand, the connection pieces are held securely on each other by the connection element in question, and, on the other hand, a degree of freedom that may still exist in a plug-in direction for connecting the connection pieces to each other is defined. In addition, the connection element can also be designed in several parts, with sub-elements that are to be connected to one another, the latter being able to be plugged in, for example.

In another development, the at least one connection element as third component can engage through the first component and the second component at least in one portion of the associated connection piece, in a manner that is easy to produce and easy to handle. This is particularly expedient in that the respective passages on the components or their connection pieces can also be subsequently introduced into these at any time. The passage on each of the components can also be designed such that the connection element only engages in these.

In a preferred development of the partial temporomandibular joint prosthesis according to the invention, passages that are flush with each other can be arranged on the connection pieces of the first component and of the second component, into which passages the at least one connection element can be inserted/plugged. The connection element can engage right through one of the connection pieces whereas it only engages in the other, or it can also completely engage through both connection pieces.

In a further development that is easy to handle, the passages of the connection pieces and the at least one connection element inserted into these are arranged substantially transverse to the direction of extent of the condyle end of the implant part. In such a configuration, the connection element can be arranged particularly easily in order to be subsequently connected to the connection pieces, if necessary. In this case, the connection element can simply be plugged through the aligned passages, in the case of sub-elements also from several sides. However, it is also conceivable that the at least one connection element inserted into the passages is arranged in a plane spanned by the movement of the first implant part, and thus substantially transverse to the configuration described above.

In another development of the partial temporomandibular joint prosthesis, a support region, arranged opposite the other connection piece, can be provided on the first and the second connection piece, which connection region supports the connection pieces during assembly, e.g. against a plug-in direction, such that they can be arranged in a predetermined, defined position relative to each other.

In a preferred development, in which changes can also be made later to the partial temporomandibular joint prosthesis, the components can be releasably connectable to each other with their connection pieces, such that components, in particular the second component with the condyle, can be easily exchanged.

In another development, which is durable, load-bearing and well tolerated, the first component with the first connection piece can be made of a titanium material.

In yet another further development of the partial temporomandibular joint prosthesis according to the invention, the second component with the second connection piece can be made of a ceramic or a thermoplastic so that it can be suitably adapted to the specific individual situation by post-processing. In the case of a ceramic second component, it can advantageously be made, for example, of a zirconium oxide material reinforced with yttrium.

In a development, the thermoplastic can preferably be made of a polyolefin such as UHMWPE (ultra high molecular weight PE) or a polyaryletherketone such as PEEK (polyetheretherketone), and the connection element is particularly preferably made of polyethylene.

The partial temporomandibular joint prosthesis according to the invention can therefore comprise a first implant part which is provided with two components, one of which is designed to carry loads and the other to roll off the movement of the jaw. The first, load-bearing component can, for example, consist of a shaft-like plate made of titanium and arranged on the mandible, at the end of which facing a joint surface there is arranged the rolling second component as a patient-specific condyle made of zirconium oxide reinforced with yttrium or UHMWPE or PEEK. Both components can be designed or adapted individually and are connected or fixed to each other via a connection element made of polyethylene, for example. Thus, the partial temporomandibular joint prosthesis according to the invention can consist of two or three materials which are firmly anchored in one another and which are not separable from one another after production, as a result of which a hybrid implant is formed. The connection of the components and of the connection element is ensured via an interlocking form-fitting surface topography, wherein the slide pairing with a possibly natural joint surface of the temporomandibular joint permits a five-axis movement.

In a preferred development, a connection element is arranged between the connection pieces, such that the connection pieces of the components are held firmly together and almost load-free fixing is achieved between the connection pieces.

In a preferred development, the connection element can be arranged between the connection pieces by a sintering process, wherein the connection element, for example a connection element arranged in the manner of a pin between the connection pieces, is baked into the assembled connection pieces in the sintering process.

In a preferred development, the connection element can be produced by compacting a plastic powder. In this case, the connection element, e.g. in powder form, can be introduced into a hole that passes through the connection pieces together, and, in the mold in which the connection pieces are placed, can first be compacted before being baking in by way of the aforementioned sintering process.

The above embodiments and developments may be combined with one another in any desired manner insofar as is rational. Further possible embodiments, developments and implementations of the invention also encompass combinations, not explicitly stated, of features of the invention that are described above or below in relation to the working examples.

### BRIEF DESCRIPTION OF THE DRAWINGS

The invention is explained in more detail below on the basis of exemplary embodiments shown schematically in the figures of the drawing, in which:

FIG. 1 shows a perspective exploded side view of a first embodiment, according to the invention, of the partial temporomandibular joint prosthesis having a first implant part with a first and second component and a connection element;

FIG. 2 shows a perspective side view of the embodiment according to FIG. 1, with first component and second component joined together;

FIG. 3 shows a further perspective side view of the embodiment according to FIGS. 1 and 2;

FIG. 4 shows an exploded perspective side view of a second embodiment of the partial temporo-mandibular joint prosthesis having a first implant part with a first and second component and the connection element;

FIG. 5 shows a perspective side view of the second embodiment according to FIG. 4, with first component and second component joined together;

FIG. 6 shows a perspective side view of a third embodiment in the implanted state, with a natural joint surface as an abutment;

FIG. 7 shows a perspective side view of a temporomandibular joint prosthesis which comprises the partial temporomandibular joint prosthesis, with an artificial second implant part, with connected components according to a fourth embodiment in the implanted state;

FIG. 8 shows an exploded perspective side view of a third embodiment of the partial temporo-mandibular joint prosthesis having a first implant part with a first and a second component and the connection element;

FIG. 9 shows a perspective side view of the third embodiment according to FIG. 8, with first component and second component joined together; and

FIG. 10a-c show side views of different steps of a production process for a method according to the invention for producing the implantable partial temporomandibular joint prosthesis.

In all of the figures, elements and devices that are the same or have the same function have been provided with the same reference signs, unless indicated otherwise.

### DETAILED DESCRIPTION

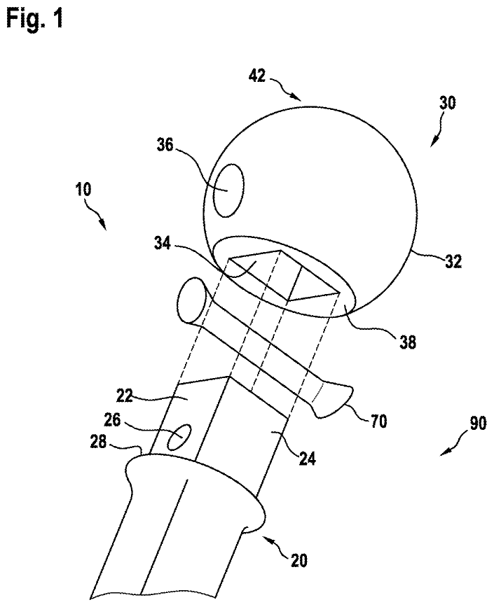

FIG. 1 shows a perspective exploded side view of a first embodiment, according to the invention, of the partial temporomandibular joint prosthesis, designated as a whole by having a first implant part 10 with a first component 20 and a second component 30 and also a connection element 70 arranged between the components 20, 30.

In the view in FIG. 1, the first component 20 is made in the form of a shaft from a titanium material and has, at its free end, an integrally formed first connection piece 22, which has a square cross section with its projection 24 in the schematic view in FIG. 1. The other end of the first component 20 is connected to the mandible, which is not shown. Opposite the first connection piece 20 is a second component 30 having a second connection piece 32 of spherical shape, which has a recess 34 facing the first component, the cross section of which recess corresponds to that of the projection 24. The second component 30 is a ceramic condyle 42. By means of the projection 24 of the first component 20 and the recess 34 of the second component 30, the first implant part 10 is releasably interconnected by the positive joining-together of the components 20, 30.

In order to hold the two components 20, 30 firmly on each other, they can be fastened to each other by a connection element 70 arranged between the respective connection pieces 22, 32. For this purpose, the connection pieces 22, 32 of the first component 20 and of the second component 30 are provided with passages 26, 36 which are flush with each other and into which the pin-like connection element 70 made of a polyethylene plastic is inserted when the components 20, 30 are in the assembled state. The passages 26, 36 and the connection element 70 inserted into them are arranged substantially transverse to the direction of extent of the condyle end of the implant part 10. Moreover, FIG. 1 shows that the two components 20, 30 are each provided, on sides facing each other, with a circumferential support region 28, 38 at which they bear on each other.

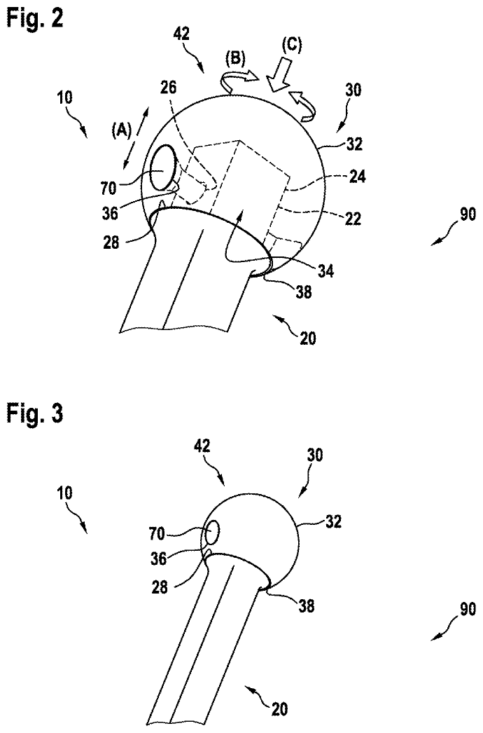

FIG. 2 shows a perspective side view of the embodiment according to FIG. 1 with first component 20 and second component 30 joined together. The arrows shown in FIG. 2 represent the forces acting on the first implant part. The arrows (A), representing forces compensating each other, show that the two components 20, 30 are fixed at their connection pieces 22, 32 almost load-free by the connection element 70, while a force applied by the form-fit engagement of the two connection pieces is taken up mainly in the direction of the arrow (C), and moreover the two components are secured against twisting relative to each other (arrows (B)).

FIG. 3 shows a further perspective side view of the embodiment according to FIGS. 1 and 2, with components 20, 30 joined together. After the components 20, 30 were joined together and placed into a sintering mold (not shown), the polyethylene pin arranged as connection element 70 between the first component 20 and the second component 30 was introduced initially as a powder into the passages 26, 36 of the components 20, 30 and compacted, in order then to be baked in a sintering process between the connection pieces 22, 32 of the components under the application of temperature and pressure.

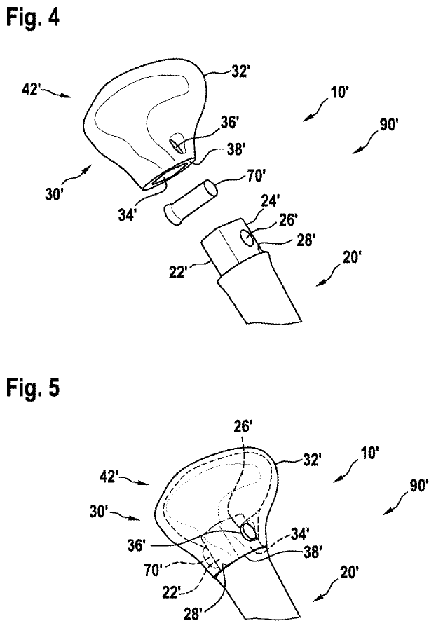

FIG. 4 shows an exploded perspective side view according to a second embodiment of the partial temporomandibular joint prosthesis 90′ having a first implant part 10′ with first and second components 20′, 30′ and also the connection element 70′. The view corresponds to FIG. 1, and therefore reference is made to the relevant statements concerning the latter. The same applies to FIG. 5, which shows a perspective side view of the embodiment from FIG. 4 with assembled first component 20′ and second component 30′, with connection element 70′ arranged between the connection pieces 22′, 32′, which corresponds to the view in FIG. 2.

The difference between the views according to FIGS. 4 and 5 and the views according to FIGS. 1 and 2 is that the former show a patient-specific first implant part 10′, i.e. one adapted to the conditions present in the patient. This is reflected on the one hand in the design of the second component 30′ with second connection piece 32′ as a specific condyle with a corresponding shape. On the other hand, the shape of the first component 20′ with the first connection piece 22′ is also adapted to the patient's mandible (not shown), in order to be able to be arranged there without any problems. Further specific adaptations involve the passages 26′, 36′ and accordingly the connection element 70′ being made shorter, the projection 24′ and the recess 34′ having rounded corner regions, and the support regions 28′, 38′ being made narrower.

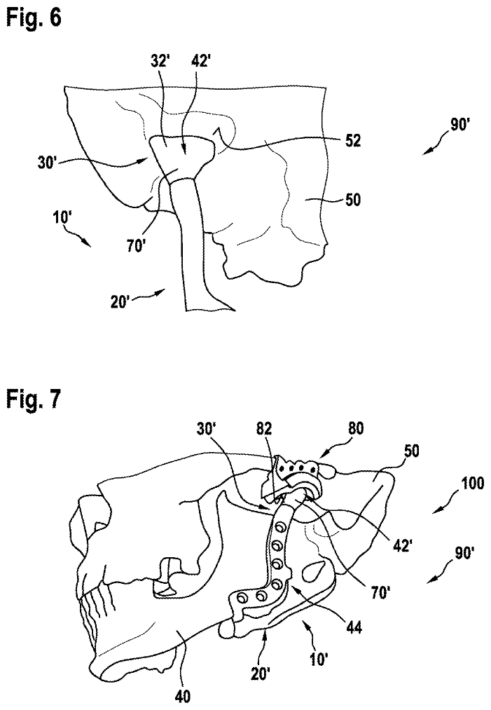

FIG. 6 shows a perspective side view of the embodiment from FIGS. 4 and 5 in the implanted state, with a natural joint surface as an abutment. The patient-specific condyle of the second component 30′ of the first implant part 10′ can be seen here, as can its first component 20′ which, for reasons of clarity, is shown without the mandible on which it is arranged. By means of the partial temporomandibular joint prosthesis 90′ according to the invention with the first implant part, a secure and defined rolling movement of the lower jaw can take place on the natural joint surface 52 of the cranium 50 shown in FIG. 6, via the condyle which is formed by the second component 30′ and whose movement is supported by the first component 20′.

FIG. 7 shows a perspective side view of a temporomandibular joint prosthesis comprising the partial temporomandibular joint prosthesis 90, with an artificial second implant part 80 which interacts with a first implant part 10 of a fourth embodiment with connected components 20, 30 in the implanted state. The first implant part 10′ is arranged on a lower jaw 40 and fastened thereto by a fastening means 44, here a hole arrangement with the pins passing through the associated holes. The free end of the first implant part assigned to the cranium 50 forms a condyle 42′, the abutment of which is formed by an artificial joint surface 82. The latter is part of a second implant part 80 arranged on the zygomatic bone of the cranium 50.

Accordingly, with the implantable temporomandibular joint prosthesis 100, which has the partial temporo-mandibular joint prosthesis 90 for the individual abutment-forming joint surface 82, it is also possible to make available a reliable slide pairing, by virtue of providing the first implant part 10 with a plurality of components 20, 30 which engage in each other with their connection pieces 22, 32 and produce a form-fit connection with these and are releasably connectable to each other.

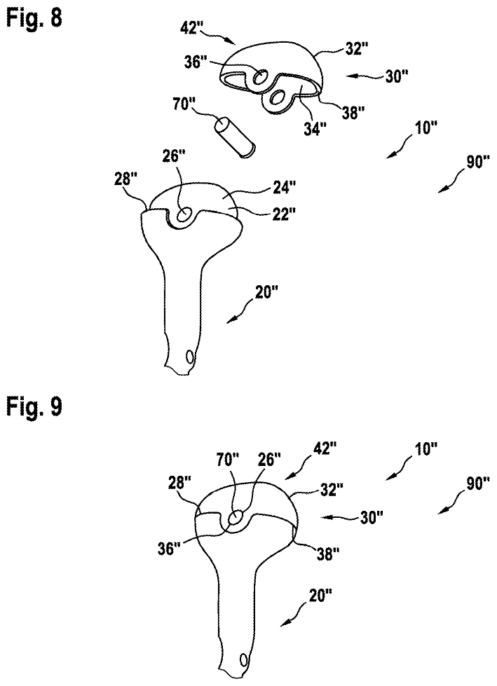

FIG. 8 shows an exploded perspective side view according to a third embodiment of the partial temporomandibular joint prosthesis 90″ having a first implant part 10″ with first and second components 20″, 30″ and also the connection element 70″, and FIG. 9 shows a perspective side view of the embodiment from FIG. 8 with assembled first component 20″ and second component 30″, with connection element 70″ arranged between the connection pieces 22″, 32″. Although the third embodiment has a greater transverse extent of the condyle 42″, reference may nonetheless also be made to the above statements concerning FIGS. 1, 2, 4 and 5 with regard to the functionality of the elements.

Looking at FIGS. 8 and 9, the embodiment of the partial temporomandibular joint prosthesis 90″ shown there, having a first implant part 10″ with first and second components 20″, 30″ and also the connection element 70″, differs from the second embodiment, shown in FIGS. 4 and 5, with regard to the configuration of the first and second components 20″, 30″ and their attachment to each other. The second component 30″ is provided here with a hood-like or shell-like second connection piece, which has the shape of half a hollow lens with the recess 34″, at the edge of which two mutually opposite extensions with passages 36″ project more or less semicircularly in the direction of the first component 20″.

This first component 20″ is provided with the first connection piece 22″, which in turn protrudes in the direction of the second component in order to engage with its projection 24″ in the recess 34″ when the two components 20″, 30″ are connected. Corresponding to the passages 36″ of the second component 30″, the passage 26″ is arranged on the first component. The connection element 70″ engages through the passages 26″, 36″ in a direction substantially perpendicular to the second embodiment, such that the longitudinal axis of the connection element 70″ in FIGS. 8 and 9 lies substantially in the plane of movement of the partial temporomandibular joint prosthesis 90″. Since the passages 36″ form part of the edge of the connection piece 32″, they are also part of the support region 38″, which comes to lie opposite a correspondingly designed support region 28″ of the first component 20″.

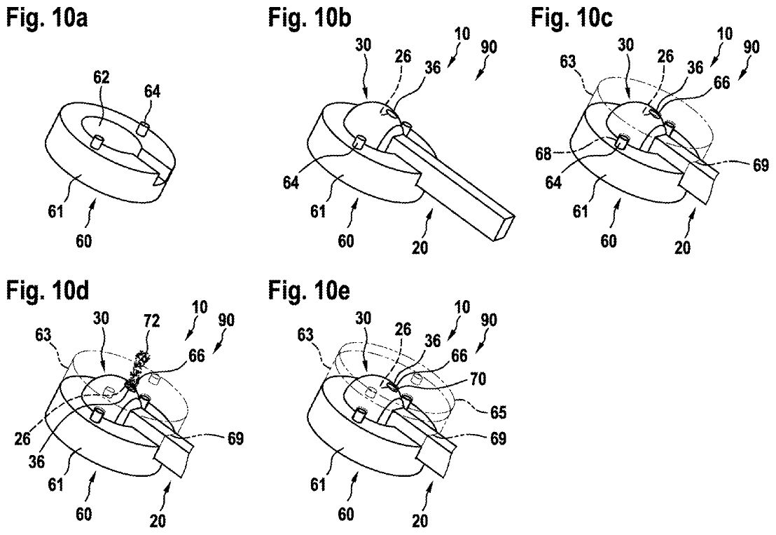

FIGS. 10a-10e show a side view of various steps of a production process for a method, according to the invention, for producing an implantable partial temporomandibular joint prosthesis 90, specifically in time sequence from FIG. 10a to FIG. 10e. FIG. 10a first of all shows a lower part 61 of a tool 60 forming a sintering mold, which has a recess 62 into which a first implant part 10 can be placed. The figure also shows pins 66 projecting from the end face of the lower part 61, which pins 66 serve for the correct positioning of a central part 63 (shown in FIGS. 10c-10e) of the tool 60.

The first implant part 10 placed in the lower part 61 of the tool 60, with its first component 20 formed by a titanium base body and with its second component 30 formed by a condyle made of zirconium, is shown in FIG. 10b. In order to securely fix the first implant part in the tool, a central part 63 is arranged thereon, the recess 69 of which central part 63 accommodates those regions of the first implant part 10 which protrude beyond the lower part 61 in FIG. 10b. As has already been mentioned, the position of the central part is determined by the pins 64 of the lower part 61, which engage in corresponding bores 68 of the central part 63. As a result, the first implant part 10 is enclosed by the tool 60 substantially on all sides at its condyle-side end region.

However, it will also be seen in FIGS. 10c and 10d that the central part 63 has a bore 66 which is flush with the passages 26, 36 of the first and second components 20, 30 of the first implant part 10. FIG. 10d shows that a third component in the form of a polyethylene powder 72 is introduced through this bore 66 between the first and the second component 20, and fills the space formed by the passages 26, 36.

After the polyethylene powder has been introduced, the tool is closed by placing a lid-like upper part 65 onto the central part 63, the polyethylene powder 72 is then compacted, and a sintering process is then started, by which the polyethylene as connection element 70 between the two connection pieces 22, 32 of the condyle, i.e. the first component and the second component 30 of the first implant part 10, is attached by being as it were “baked in” between the components 20, 30.

Although the present invention has been described above on the basis of preferred exemplary embodiments, it is not limited thereto and instead can be modified in a variety of ways. In particular, the invention can be changed or modified in many ways without departing from the core of the invention.

1. An implantable partial temporomandibular joint prosthesis having:
a first implant part which can be fastened to a lower jaw and has a condyle with respect to which a joint surface arranged on a cranium forms an abutment;
wherein the first implant part has a first component made of a metal material, which first component can be fastened to the lower jaw, and a second component made of a ceramic or a plastic, which second component has the condyle;
wherein the first component is provided with a first connection piece and the second component is provided with a second connection piece, and the first connection piece and the second connection piece are connectable to each other by form-fit engagement at least in part.

2. The partial temporomandibular joint prosthesis as claimed in claim 1, wherein one of the first and second connection pieces is designed with includes at least one projection which is configured to engage in at least one complementary recess on the other of the first and second connection pieces.

3. The partial temporomandibular joint prosthesis as claimed in claim 2, wherein the at least one projection of the one of the first and second connection pieces and the at least one complementary recess of the other of the first and second connection pieces are configured to produce an all-round form fit.

4. The partial temporomandibular joint prosthesis as claimed in claim 1, wherein the first component and the second component are fastened to each other by at least one connection element arranged between the respective first and second connection pieces.

5. The partial temporomandibular joint prosthesis as claimed in claim 4, wherein the at least one connection element engages through the first component and the second component at least in a portion of the respectively associated first and second connection pieces.

6. The partial temporomandibular joint prosthesis as claimed in claim 4, wherein passages flush with each other are arranged on the first and second connection pieces of the first component and of the second component, into which passages the at least one connection element is insertable.

7. The partial temporomandibular joint prosthesis as claimed in claim 6, wherein the passages and the at least one connection element inserted therein are arranged substantially transverse to the direction of extent of the condyle end of the implant part.

8. The partial temporomandibular joint prosthesis as claimed in claim 1, wherein, the first and the second connection pieces, each include a respective protection region in which the respective protection regions are arranged opposite each.

9. The partial temporomandibular joint prosthesis as claimed in claim 1, wherein the first connection piece and the second connection piece are releasably connectable or connected to each other.

10. The partial temporomandibular joint prosthesis as claimed claim 1, wherein the first component with the first connection piece is made of a titanium material.

11. The partial temporomandibular joint prosthesis as claimed in claim 1, wherein the second component with the second connection piece is made of a ceramic or a thermoplastic.

12. The partial temporomandibular joint prosthesis as claimed in claim 11, wherein the thermoplastic is made of a polyolefin or a polyaryletherketone.

13. The partial temporomandibular joint prosthesis as claimed in claim 4, wherein the connection element is made of polyethylene.

14. A method for producing an implantable partial temporomandibular joint prosthesis having:
a first implant part which can be fastened to a lower jaw and has a condyle with respect to which a joint surface arranged on a cranium forms an abutment;
wherein the first implant part has a first component made of a metal material, which first component can be fastened to the lower jaw, and a second component made of a plastic, which second component forms the condyle;
wherein the method comprises at least the following steps:
providing the first component with a first connection piece;
providing the second component with a second connection piece;
joining the first and second connection pieces together with a form fit, with application of temperature and vacuum.

15. The method as claimed in claim 14, further comprising attaching a connection element between the connection pieces.

16. The method as claimed in claim 15, wherein the connection element is arranged between the connection pieces by a sintering process.

17. The method as claimed in claim 15, wherein the connection element is produced by compacting a plastic powder.

18. An implantable temporomandibular joint prosthesis, comprising the partial temporomandibular joint prosthesis with the first implant part as claimed in claim 1 produced by a method as claimed in claim 13, said implantable temporomandibular joint prosthesis having a second implant part with a joint surface which can be arranged on the cranium and which forms the abutment with respect to the condyle.
